(19) United States
(12) Patent Application Publication
(10) Pub. No.: US 2022/0183992 A1
(43) Pub. Date: Jun. 16, 2022

(54) Edible Film Comprising Adjacent Conjoined Strips

(71) Applicant: Terpene Therapeutics Inc., Ottawa (CA)

(72) Inventor: Michael Bousfield, Burlington (CA)

(21) Appl. No.: 17/436,354

(22) PCT Filed: Mar. 6, 2020
(86) PCT No.: PCT/CA2020/050305

§ 371 Date: Sep. 3, 2021

Related U.S. Application Data
(63) Continuation-in-part of application No. 16/295,554, filed on Mar. 7, 2019, now abandoned.

(51) Int. Cl. A61K 9/70; A61K 9/00; A61K 45/06

(52) U.S. Cl. CPC A61K 9/7007; A61K 45/06; A61K 9/006; A61K 9/0056

(57) ABSTRACT

Edible terpene-containing films are provided comprising at least one terpene or terpenoid, at least one film-forming agent, at least one surfactant or emulsifier, and a plasticizer. The films may be coated with a buccal and/or sublingual muco sal absorption enhancer, an anti-microbial agent and/or comprise at least two adjacent conjoined strips having a distinct functionalities.

### FIELD OF THE INVENTION

The present invention generally relates to edible films, and more particularly relates to edible films comprising adjoined or conjoined strips.

### BACKGROUND OF THE INVENTION

Edible film products are known in the art. They were traditionally provided in simple strip form, but have been modified over the years to assume, for example, different shapes, layers, colours and/or flavours.

Edible oral care strips that provide a breath-freshening function have been in existence for some time, and typically comprise a film-forming agent and a flavor agent such as menthol, peppermint or spearmint.

Novel forms of consumable edible thin films have more recently been developed as described, for example, in US Patent Application No. 2004/0096569 to Barkalow et al. that vary in color, flavor, shape and mouthfeel, to improve the marketability of edible films to younger consumers.

US 2003/0224090 describes edible films as snacks devised to provide flavor and/or craving satisfaction. These films comprise high levels of appealing flavors and flavor combinations, as well as sweeteners and other ingredients. The snack may include one or more layers to preserve structure, or to enhance flavor sensation.

Despite the foregoing, it would be desirable to develop novel edible films.

### SUMMARY OF THE INVENTION

Novel edible films are herein provided comprising adjoined or conjoined strips.

Thus, in one aspect of the invention, an edible film is provided comprising at least one terpene or terpenoid, at least one film-forming agent, a surfactant and/or emulsifier and a plasticizer, wherein said film comprises at least two conjoined strips, each strip having a distinct functionality.

In another aspect of the invention, an edible film is provided comprising at least one terpene or terpenoid, at least one film-forming agent, a surfactant and/or emulsifier and a plasticizer, wherein the film is coated with a buccal and/or sublingual mucosal absorption enhancer and/or an anti-microbial agent.

These and other aspects of the invention are described by reference to the following figures.

### BRIEF DESCRIPTION OF THE FIGURES

FIG. 1 illustrates the terpene profile of the Blue Dream strain;

FIG. 2 illustrates the terpene profile of the Granddaddy Purple strain;

FIG. 3 illustrates the terpene profile of the Girl Scout Cookies strain;

FIG. 4 illustrates the terpene profile of the Green Crack strain;

FIG. 5 illustrates the terpene profile of the OG Kush;

FIG. 6 illustrates the terpene profile of the Sour Diesel strain;

FIG. 7 illustrates properties of terpenes of different classes; and

FIG. 8 illustrates a film in accordance with an embodiment of the invention.

### DETAILED DESCRIPTION OF THE INVENTION

An edible film is provided comprising at least one terpene or terpenoid, at least one film-forming agent, at least one surfactant or emulsifier, and a plasticizer, wherein the film comprises at least two adjacent adjoined or conjoined strips, each strip having a distinct functionality.

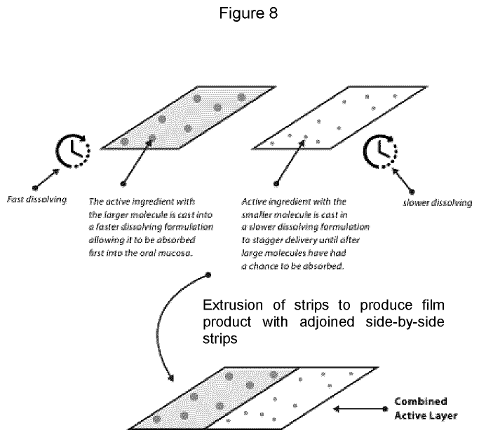

The film comprises at least two adjacent adjoined or conjoined strips having distinct functionalities. The term “adjoined” or “conjoined” as it relates to adjacent strips refers to strips which are joined together or contiguous. The term “adjacent” is used herein may be used to refer to any adjacent arrangement. In a preferred embodiment, the term adjacent is used to refer to adjoined strips within a film which are in a side-by side or edge to edge arrangement, i.e. the edge of a longitudinal side of a first strip of a film borders and is joined to the edge of a longitudinal side of a second strip as shown in FIG. 8. The term “functionality” is used herein to refer to the utility or function of a strip. Functionality may include dissolution properties, delivery properties, adhesion properties, therapeutic properties, flavor, etc. The functionality of the strips of a film may relate to the same property or different properties. For example, a film may be provided with two or more conjoined strips in which each strip exhibits a different dissolution rate, in which each strip exhibits different adhesion properties, or in which each strip has different doses of the same compound or therapeutic with different dissolution rates. Alternatively, a film may be provided with two or more conjoined strips having unrelated functionalities, for example, a film with two or more conjoined strips each having different therapeutic or other utility, or a film with a flavor strip and a therapeutic strip which may have the same or different dissolution rates.

The film may comprise any edible film forming agent including, but not limited to, pullulan, sodium alginate, carrageenan, methyl cellulose, ethyl cellulose, hydroxypropylmethyl cellulose, hydroxypropyl cellulose, corn starch, pectin, chitin, chitosan, collagen, gelatin, zein, gluten, cassava starch, glucan and mixtures thereof.

The film forming agent may be used alone or combined with one or more agents that facilitate film forming, including but not limited to, one or more of hydroxyethyl cellulose, polyvinyl pyrrolidone, carboxymethyl cellulose, polyvinyl alcohol, polyethylene glycol, polyacrylamides, glycolide, polylactic acid, polylactide, cellulose gum, xanthan gum, tragacanth gum, guar gum, acacia gum, arabic gum, gum karaya, welan gum, polyacrylic acid, sodium methacrylic acid polymers, methacrylic acid copolymers, acrylic acid polymers, acrylic acid copolymers, methylmethacrylate copolymer, carboxyvinyl polymer, amylose, high amylose starch, casein, algin, maize, chondrus crispus, crosspovidone (CPV), polyvinylpyrrolidone (PVP), hydroxymethylcellulose, glycogen, phytoglycogen, polysaccharides, hydroxypropylate, dextrin, maltodextrin, soy protein isolate, whey protein isolate, beeswax, carnauba wax, fungal exopolysaccharides, rapeseed, and mixtures thereof.

The film forming agent, or combination of film-forming agents, is present in an amount ranging from about 0.01 to about 99 wt %, preferably from about 30 to about 80 wt%, more preferably from about 45 to about 75 wt % of the film, and even more preferably, from about 60 to about 65 wt % of the film. A preferred film forming agent is pullulan.

In one embodiment, the present film may comprise one or more fast-dissolving strips in which the film forming agent is selected from pullulan, sodium alginate, and mixtures thereof to result in a film that dissolves in 5-60 seconds as measured using a dissolve rate test such as a permeation test that mimics the oral environment. In an example, quick dissolving film strips comprise pullulan in an amount ranging from 1 to 99%, optionally combined with sodium alginate, in an amount ranging from about 1 to 99% is provided. The strips may exhibit different dissolution rates by altering the film forming agent content of the strips. For example, by using a fast dissolving film forming agent, or a greater content of a fast dissolving film forming agent such as pullulan, the dissolution rate of the film can be increased, while using a film forming agent that dissolves at a slower rate, e.g. alginate, or a greater content of such a film forming agent, the dissolution rate of the film is decreased. Dissolution may also be altered by adjusting the pH of the film, for example, increasing the pH of the film, will generally increase dissolution rate of the film.

In another embodiment, the present film may comprise one or more slow dissolving strips in which the film forming agent is selected from hydroxypropyl methylcellulose, carrageen, and mixtures thereof, additionally comprising one or more facilitating agents such as maltodextrin to result in a film that dissolves in 61-180 seconds according to a permeation test. By altering the film forming agent content of a strip, different dissolution rates may be attained. For example, a slow dissolving film may be attained which comprises hydroxypropyl methylcellulose (HPMC), carrageen and maltodextrin in amounts ranging from 11-45% by wt HPMC, 1-23% by wt carrageen and 5-29% by wt maltodextrin. Higher amounts of HPMC would result in a slower dissolving strip, while higher amounts of sodium alginate would result in a less slow dissolving strip.

Any suitable method may be used to determine dissolve rate of a film. An exemplary method includes tautly fastening a sample of the film (preferably, 30 to 35 mm long, 20-25 mm in width and a thickness of 0.053mm-0.063 mm) with adhesive tape on the test plate of a dissolution testing apparatus. A chrome ball bearing (about 6.35 mm in diameter, and 1.045+/−0.002 g) is placed centrally on top of the film and 2 drops of water at ambient temperature are dropped onto the top of the ball bearing. The time for the ball to drop to the test plate 10 mm below represents the dissolution rate. The longer the time, the slower the dissolution rate. This method is referred to herein as EI QC 019.

A film may be provided comprising one or more fast-dissolving strips as well as one or more slow dissolving strips. Thus, the film may comprise a strip or strips comprising pullulan, sodium alginate, or mixtures thereof to form a fast-dissolving strip, and a strip or strips comprising hydroxypropyl methylcellulose, carrageen, or mixtures thereof to form a slower-dissolving strip or a strip that dissolves at a reduced rate. The provision of a film comprising strips that dissolve at different rates permits staged delivery of the content of the film, such as any active ingredients present therein. For example, in a film comprising adjacent adjoined strips, a terpene or other active ingredient or combination of active ingredients may be contained in a first fast-dissolving strip comprising a buccal absorption enhancer to promote increased buccal mucosal uptake, and thereby reduce the amount of active ingredient that bypasses mucosal uptake. A terpene or other active ingredient or combination of active ingredients may be contained in a second slower-dissolving strip in order to allow the buccal absorption enhancer released in the first fast-dissolving strip to have an effect on the buccal membrane before release of the active ingredient(s) from the second slower-dissolving strip, thereby resulting in increased absorption of the active ingredient(s) in the second strip on release from the film.

The present film will generally also include a surfactant, i.e. a compound that lowers the surface tension (or interfacial tension) between two liquids or between a liquid and a solid. Examples of surfactants include, but not limited to, glycerol, glycerin, propylene glycol, sucrose fatty acid ester, sorbitan monostearate, sorbitol-based surfactants such as polysorbate 60 or 80, lecithin such as soy or sunflower lecithin, modified cellulose gum (CMC), ceratonia gum (locust bean gum), sodium lauryl sulfate (SLS), or mixtures thereof. The film will generally comprise a surfactant in an amount ranging from about 0.5 to 15% by wt, e.g. 0.5 to 12.7 wt %, of the film.

An emulsifier is a type of surfactant, and functions to stabilize an emulsion. The film may include an emulsifier in an amount of about 1% to 15% by wt of the film. Examples of suitable emulsifiers include, but are not limited to, polyoxyethylene, sorbitan monostearate, lecithin such as soy or sunflower lecithin, stearate salt such as potassium stearate, calcium stearate or sodium stearate, calcium stearoyl lactylate (CSL), glyceryl monostearate, mono propylene glycol, sorbitan monooleate (SPAN 80), sorbitol-based emulsifiers such as polysorbate 60 or 80 (Tween 60/80), monoglycerides and diglycerides (e.g. glycerol monostearate) glycerol triacetate and mixtures thereof.

The film may include a surfactant as well as an emulsifier, for example, sorbitol-based surfactants, such as polysorbate 60 or 80, glycerin or propylene glycol, combined with emulsifiers such as sorbitan monostearate, a stearate salt, or polyoxyethylene.

The film will generally also include plasticizer that increases the plasticity and/or decreases the viscosity of the film. Examples of plasticizers include, but are not limited to, monostearate palm oil, lethicin, sorbitan monostearate, glycerin, glycerin fatty acid ester, triacetin, pre-gelatinized starch, sorbitol, medium chain mono- or diglycerides, castor oil, malic acid, polyethylene glycol (PEG), glycerol, palm oil and mixtures thereof. The film will generally comprise plasticizer in an amount ranging from about 2 to 18 wt % of the film.

It is noted that some compounds may have multiple functions, and thus, satisfy multiple roles in the present film, for example, sorbitol-based compounds function as both a surfactant and an emulsifier, sorbitan monostearate and lecithin function as a surfactant, emulsifier and plasticizer, and glycerol functions as both a surfactant and a plasticizer.

The film may include a sweetener in an amount in the range of 0% to 11% by wt. Examples of suitable sweeteners include but are not limited to: sucralose, acesulfame potassium, aspartame, stevia/rebaudioside, xylitol, neotame, saccharin, erythritol, isomalt, maltitol, lactitol, mannitol, glycerol (glycerine), glycyrrhizin and mixtures thereof.

The film may include a flavouring agent in an amount ranging from 0.1% to 15% by wt of the film. Examples of flavours include, but are not limited to, peppermint, strawberry, cherry, menthol, vanillin, fenugreek seed extract, spearmint, raspberry, ginger, pomegranate, passion fruit, peach, orange, tutti fritti, cola, honeydew, yuzu, cinnamon, mango, kumquat, lychee, grapefruit, spice extracts, and terpenes, e.g. limonene, myrcene, etc.

The present film comprises at least one terpene or terpenoid in an amount ranging from about 0.01% to 30% by wt of the film. The term “terpene” or “terpenoid” refers to hydrocarbons derived from units of isopentenyl pyrophosphate and to functionally equivalent derivatives thereof. Examples of terpenes include myrcene, beta caryophyllene, pinene, limonene, terpinolene, humulene, nerolidol, linalool, ocimene, guaiol, bisabolol, alpha phellandrene, cadinene, camphene, camphor, citral, citronellol, delta 3-carene, eucalyptol, eugenol, gamma terpinene, geraniol, nerol, ocimene, paracymene, phytol, pulegone, terpineol, valencene, functionally equivalent analogues or derivatives thereof such as oxides thereof. The term “functionally equivalent” as it relates to analogs and derivatives of a terpene refers to compounds which exhibit the same or similar activity or effect, e.g. at least about 50% of the activity of the terpene from which it is derived.

Terpenes may be classified by the number of molecular isoprene units. Thus, terpenes herein include hemiterpenes having a single isoprene unit such as isoprene and oxygen-containing derivatives such as prenol and isovaleric acid; monoterpenes having two isoprene units and the molecular formula Cioffi6 such as geraniol, terpineol, limonene, myrcene, ocimene, terpinolene, pinene and iridoids which are derived from monoterpenes; sesquiterpenes having three isoprene units and the molecular formula C₁₅H₂₄, such as humulene, caryophyllene, farnesenes, farnesol, B-bisabolene, B-curcimene, lanceol, juvabione and humbertiol; diterpenes having four isoprene units and the molecular formula C₂OH₃₂ derive from geranylgeranyl pyrophosphate and include cafestol, kahweol, cembrene and taxadiene (precursor of taxol); sesterterpenes having 25 carbons and five isoprene units, such as geranylfarnesol; triterpenes having six isoprene units and the molecular formula C₃₀H₄₈ such as squalene;

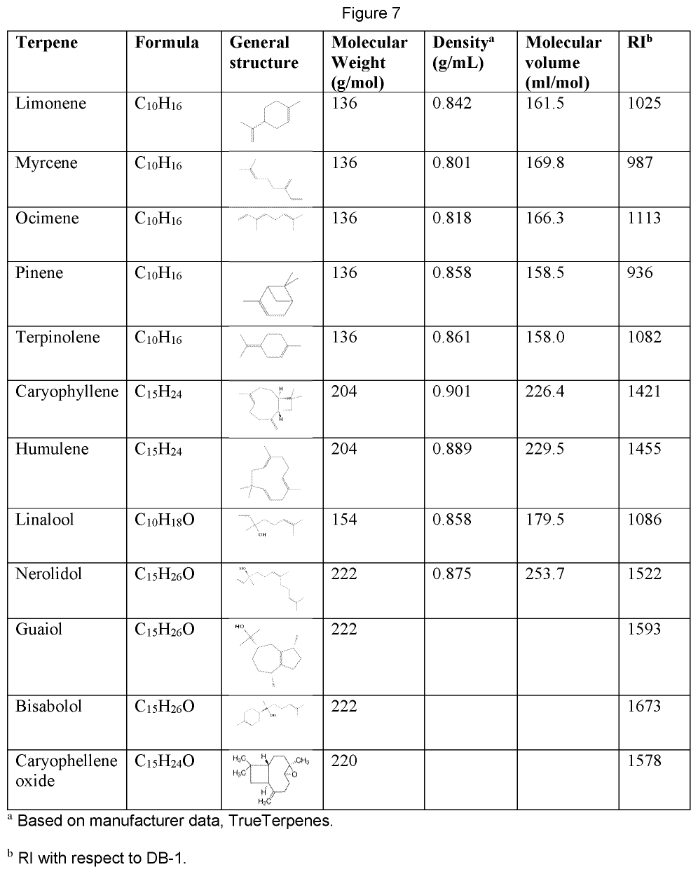

sesquarterpenes having seven isoprene units and the molecular formula C₃₅H₅₆, such as ferrugicadiol and tetraprenylcurcumene; and tetraterpenes having eight isoprene units and the molecular formula, C₄₀H₆₄, such as acyclic lycopene, the monocyclic gamma-carotene, and the bicyclic alpha- and beta-carotenes. Terpenes may also be classified as terpene derivatives or analogues such as hydroxylated terpenes such as linalool, nerolidol, guaiol and bisabolol; and oxygenated terpenes such as caryophyllene oxide and limonene oxide. The properties of some of the terpenes within these classes are shown in FIG. 7. Preferential terpene release can be fine-tuned based on differences or similarities thereof, such as molecular volume and retention index (RI).

Terpenes may be extracted from various plant sources. Terpenes may also be chemically synthesized. Preferred terpenes for use in the present film product are purified terpenes obtained from various sources, preferably obtained from non-Cannabis sources.

The film may also include one or more additional active ingredients in an amount in the range of about 0.01% to 15% by wt of the film, such as nutraceutical, pharmaceutical or therapeutic agents, including, but not limited to, botanical and/or herbal extracts, vitamins and minerals, pain medication (such as acetaminophen, non-steroidal anti-inflammatory agents (NSAIDS) such as aspirin, ibuprofen and diclofenac, anti-bacterial agents, decongestants, antihistamines, psychological treatments (e.g. anti-depressive and anti-anxiety agents), gastrointestinal treatments, sleep disorder treatments, cannabinoids, and mixtures thereof.

The term “cannabinoids” is meant to encompass naturally occurring cannabinoids such as phytocannabinoids and endocannabinoids (such as arachidonoyl-ethanolamide (anandamide), 2-arachidonoyl glycerol (2-AG) and arachidonyl glyceryl ether (noladin ether)), synthetic cannabinoids (manufactured artificially), and functionally equivalent derivatives and analogues of any of these. Examples of cannabinoids include, but are not limited to, cannabidiol (CBD), cannabidiol acid (CBDA), cannabinol (CBN), cannabigerol (CBG), cannabigerol acid (CBGA), cannabidivarin (CBDV), cannabidivarin acid (CBDVA), cannabinovarin (CBNV), cannabigerovarin (CBGV), cannabichromene (CBC), CBCV (cannabichromevarin), naphthoylindoles such as JWH-018, JWH-073, JWH-398, JWH-200, JWH-081, 4-methyl-JWH-073, JWH-015, JWH-122, JWH-220, JWH-019, JWH-007; phenylacetylindoles such as JWH-250 and JWH-203; benzoylindoles such as RCS-4, AM-694 and WIN 48,098; cyclohexylphenoles such as CP 47,497-C8 and CP 47,497; and HU-210. Cannabinoids also include tetrahydrocannabinoid and analogs thereof, namely, delta-9 tetrahydrocannabinol (THC) and functionally equivalent compounds, including analogs and derivatives thereof such as delta-8 tetrahydrocannabinol (D8-THC), tetrahydrocannabinol acid (THCA), tetrahydrocannabivarin (THCV), tetrahydrocannabivarin acid (THCVA), nabilone, rimonabant (SR141716), JWH-018, JWH-073, CP-55940, dimethylheptylpyran, HU-210, HU-331, SR144528, WIN 55,212-2, JWH-133,1evonantradol and AM-2201. The term “functionally equivalent” as it relates to analogs and derivatives of a cannabinoid refers to compounds which exhibit the same or similar therapeutic effect, e.g. at least about 50% of the activity of the cannabinoid from which it is derived. Cannabinoids may be extracted from the cannabis plant using methods well-established in the art. Many of the cannabinoids may also be prepared using standard chemical synthetic methods. Some of these compounds are also commercially available.

The film may additionally contain a mucosal permeation or absorption enhancer, e.g. sublingual and/or buccal permeation enhancer, to aid in delivery of the terpene and any other active ingredients. An absorption enhancer in an amount in the range of about 0.01 to 5% by wt of the film may be included in the film. Examples of absorption enhancers that may be used include, but are not limited to, 23-lauryl ether, aprotinin, azone, taurine, dextran sulfate, cetylpyridinium chloride, lauric acid, menthol, xylitol, oleic acid, polyoxyethylene, polysorbate 80, salts of EDTA, deoxycholate, glycocholate, lauryl sulfate, glycodeoxycholate, salicylate, taurocholate and dodecyl sulphate (e.g. sodium salts), palmitoyl carnitine and alkyl glycosides. Terpene absorption enhancers may also be used including alpha-limonene, dextro-limonene, nerolidol, menthol, alpha-bisabolol and eucalyptol.

An anti-oxidant may also be included in the film to prevent or reduce oxidation of the active ingredients in the film. Examples of suitable anti-oxidants include, but are not limited to, sodium thiosulfate, ascorbic acid, fatty acid esters of ascorbic acid, alpha-tocopherol, propyl gallate, erythorbic acid, sodium erythorbate, citric acid, tocopherols and alkyl-substituted paraben or salts thereof such as ethyl paraben, methyl paraben, propyl paraben or sodium salts thereof. Antioxidant may be included in the present film in an amount suitable to reduce or prevent oxidation, for example, an amount in the range of about 0.01 to 2.5% by wt of the film.

In one embodiment, the adjacent conjoined strips of the present film each comprise a different terpene or combination of terpenes, or may contain in one strip a terpene or terpenes and one or more pharmaceutical, nutraceutical or therapeutic agents in adjacent strips in amounts (dosages) sufficient for their function.

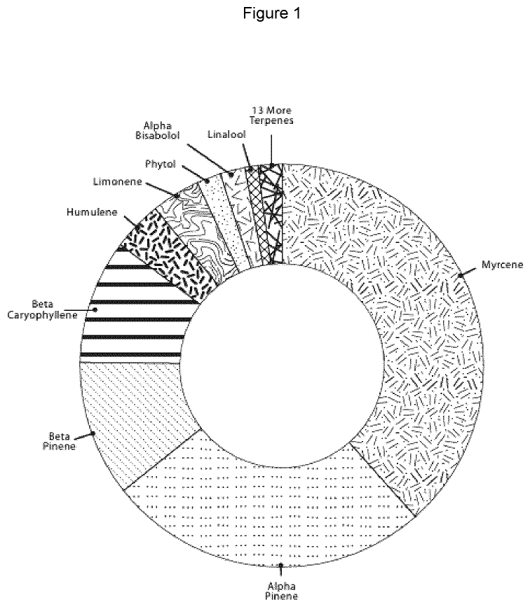

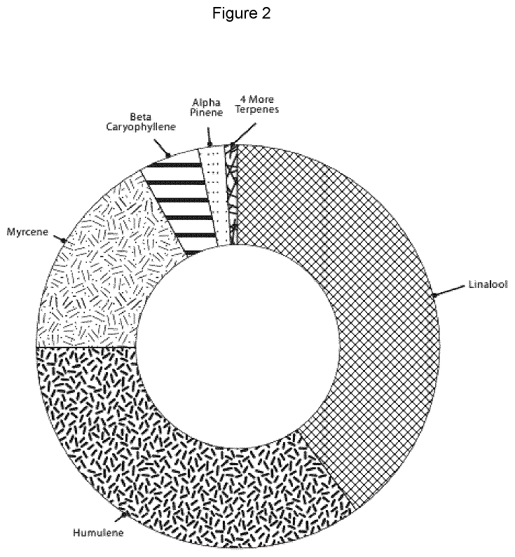

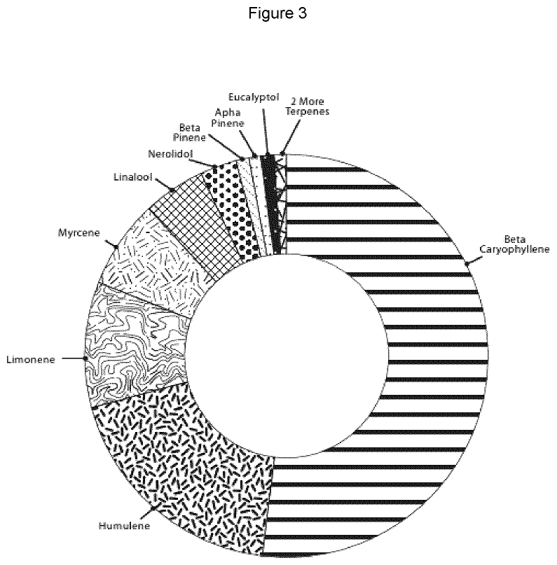

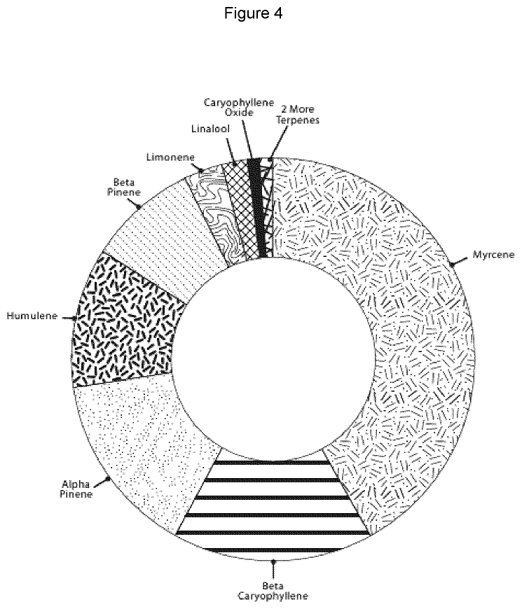

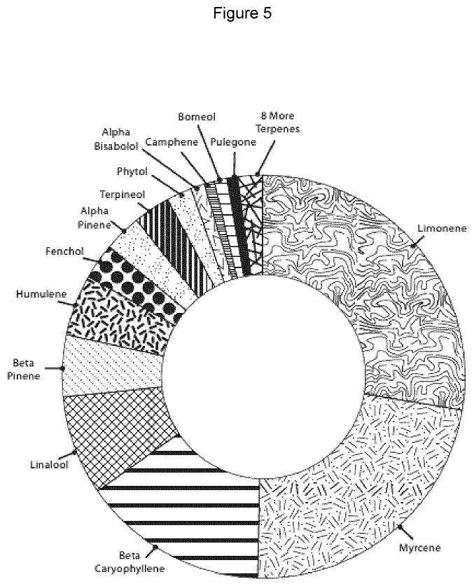

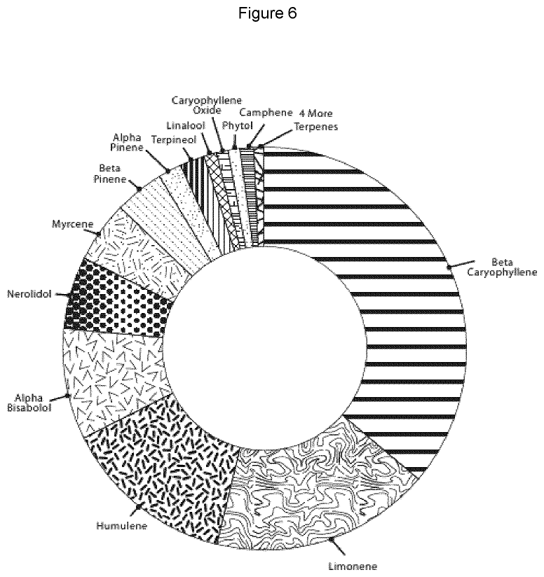

For example, the present film may include two or more strips to mimic the terpene content or profile, and optionally the cannabinoid content, of naturally occurring Cannabis strains such as Blue Dream, Bubba Kush, Granddaddy Purple, Girl Scout Cookies, Green Crack, Jack Herer, OG Kush, Sour Diesel, White Widow, GG#4, SFV OG, Lemon Skunk, Gelato, Strawberry Banana, Orange Cookie, Cherry Pie, Banana Kush, Wifi, Zkittles, Goji, Sativa Blend, Pineapple Express, Indica Blend, Head Band, Durban Poison, Dog Walker OG, ATF, ACDC, Clementine, Tangie, Sunset Sherbet, Trainwreck, XJ-13, Tahoe OG, Super Lemon Haze, Blue Cheese, Mimosa, Do-si-dos, Sundae Driver, Superglue and Watermelon. FIGS. 1-10 illustrate the terpene profiles/content of some Cannabis strains.

Regarding such terpene-containing films, these films may include strips with the complete terpene content of a Cannabis strain, may include strips with only the major terpenes of a Cannabis strain, or may include a single or combination of terpenes to yield a certain effect, including a certain psychoactive effect such as drowsiness, energy boost, euphoria, relaxation and calming, or a therapeutic effect. Certain terpenes are useful to treat anxiety and depression such as limonene, linalool, and beta caryophyllene. Others are useful as a sleep aid such as myrcene and terpinolene. Others have anti-inflammatory properties such as pinene and beta caryophyllene, and others have anti-tumour properties such as limonene and humulene.

Examples of particular terpene strip combination films having therapeutic utility include a film useful to treat gastrointestinal discomfort comprising limonene, beta caryophyllene, and optionally, gingerol; a film useful to treat anxiety comprising linalool and myrcene, a film useful to treat depression comprising beta caryophyllene and limonene, a film useful to treat inflammation comprising pinene and beta caryophyllene, a film having anti-cancer utility comprising limonene, humulene and guaiol terpene, a film useful to provide an energy boost comprising pinene and eucalyptol, a calming or anti-stress film comprising linalool and myrcene, terpineol, borneol or nerolidol, a sleep-inducing film comprising terpinolene and myrcene, or a pre- or post work-out strip comprising pinene and eucalyptol, and optionally beta caryophyllene, linalool or myrcene. As set out previously, the strips of a film may each include single terpenes, or may include 2 or more terpenes per strip, or any combination thereof.

The present films may also comprise one or more terpene-containing strips and one or more strips comprising another active ingredient that complements the terpene content. Thus, the strips of a film may comprise complementary anti-inflammatory treatments (e.g. NSAID or acetaminophen with pinene and/or beta caryophyllene); complementary psychological treatments (e.g. anti-depressive and anti-anxiety agents) with terpenes such as linalool and myrcene (anti-anxiety) or beta caryophyllene and limonene (anti-depression), sleep disorder treatments with terpinolene and/or myrcene; a combination of herbal and/or botanical extracts with selected terpenes such as a combination of limonene, beta caryophyllene, and optionally, gingerol with ginger, sabinene and balsam to treat gastrointestinal discomfort; kana and kava with linalool and myrcene, terpineol, borneol or nerolidol for anti-stress, or with beta caryophyllene and limonene for anti-depression; and the like.

Generally, the present films are made by blending the dry ingredients (e.g. film-forming agents) together and mixing with liquid ingredients including water, other solvents and/or an aqueous phase including water soluble ingredients, to form a homogeneous liquid blend. The film is then extruded/cast and coated onto a moving belt or drum for drying/cutting/rolling. The drying phase may include, for example, air drying, baking, vacuum drying, microwave drying, radio-frequency drying or dehydrating with circulating warm air. Temperature for film extrusion is in the range of about 20-50 ° C. to produce a film having a moisture level in the range of about 8.5-10%, preferably about 9%. Drying conditions include a temperature of about 40-105° C. and a relative humidity of about 10-60%.

As one of skill in the art will appreciate, slot extrusion techniques are useful to achieve uniform extrusion of the film, and may also be adapted to extrude different films adjacent to one another to yield a film comprising two or more distinct adjacent conjoined strips. In one embodiment, two or more different films are extruded in a side-by-side arrangement to yield a film comprising two or more strips joined along the length of a bordering edge as shown in FIG. 8. In this regard, multiple extruders and specially constructed dies may be useful as described in US 2004/0096569. Generally, a film-forming slurry (or slurries) is fed into a hopper (or hoppers) that channels the slurry (or slurries) for extrusion through a selected arrangement of extruder(s) and dies to achieve the desired conjoined strip-containing film.

In one embodiment, a slurry for each strip of the film may be prepared as follows. The dry ingredients (such as film-forming agents, plasticizer and active ingredients) are blended together, combined with potable water that has a total dissolved solid content of lower than 300 parts per million, and mixed with a high shear mixer (at about 45-55 rpm) until blended (for 10-60 minutes) at a temperature of about 65-80° C. Liquid ingredients are then added (such as surfactant/emulsifier and flavor agent) and high shear mixing is continued for 10-60 minutes at about 45-55 rpm to form a homogeneous blend. Once mixing is complete, the resulting slurry is allowed to rest for a period of time such as about two hours. Following resting, the slurry is then extruded/cast onto a moving belt, drum or mylar web which passes through a series of heating (about 40-105° C.) and cooling cycles to result in a film with a 8.5-9.5% homogeneous moisture level, preferably about 9%, and a thickness of about 0.001″ to 0.020″ (e.g. 25 to 635 microns).

The present films provide several advantages. For example, the present films readily permit the preparation of an orally administrable product comprising a selected combination of active ingredients, including one or more selected terpenes with or without one or more additional pharmaceutical, neutraceutical and/or therapeutic agents. As described herein, the preparation of the present edible film, customized to include select components, e.g. select terpenes and other components in precise amounts, can be conducted expediently (e.g. in a number of hours), and thus, the flexibility to be able to readily provide a modified film to exclude one or more ingredients, add additional ingredients, or alter the amounts of the ingredients, exists. Since the present films enable the provision of a product comprising a particular selected terpene content, films with a unique terpene content may be prepared to achieve a certain targetted effect, including a psychoactive effect or a therapeutic effect or a combination thereof

The present films also enable the provision of a product that mimics an existing Cannabis product, in terms of content and effect, but which is prepared with purified terpenes, including terpenes from non-Cannabis sources. This significantly accelerates the process to provide such Cannabis-like products in comparison to the provision of a natural Cannabis product, is useful in jurisdictions where Cannabis is not legal, and avoids the constraints associated with cultivating Cannabis plants (e.g. cost, substantial growing times, plant variation, etc). or the constraints of using extracts from cultivated Cannabis plants (e.g. fixed terpene content and amounts). Thus, a means that readily allows the preparation of films with various selected terpenes and various selected amounts of terpenes on demand is provided. The present film product, thus, enables the use of terpenes extracted from a vast array of non-Cannabis plant sources to provide a product that possesses a consistent terpene makeup that may mimic the content of various Cannabis strains, or a product which is altered from such strains as desired, e.g. with or without CBD and or THC. The present film, thus, provides a cost effective alternative to cultivating Cannabis.

The provision of a film product comprising distinct strips also provides flexibility with respect to the combination of ingredients therein, avoiding the potential for any undesirable interaction between the terpene or other active ingredients during production or post-production. In other words, terpenes or other active ingredients known to interact in an undesirable way may be prepared in separate formulations (i.e. slurries) and subsequently be included in the same final film product as distinct strips in which the contents will have little interaction. This will result in an increased shelf-life of the film product providing a product in which potentially reactive components, e.g. terpenes, are separated and prevented from interacting.

Similarly, the present film provides flexibility when working with different terpenes having different properties, such as different flash points. For example, the flash point for beta-caryophyllene is 214° F., and the flash point for limonene is 119° F., the flash point for alpha pinene is 91° F., the flash point for linalool is 172° F. and the flash point for myrcene is 103° F. Thus, the provision of the present film product having distinct strips permits handling of different terpenes under suitable conditions for each terpene so as to account for their different flash points. In other words, a terpene having a high flash point may be formulated in a slurry for inclusion as a first strip in the present edible film under appropriate conditions and separately from the formulation of a terpene having a low flash point which itself may be separately formulated under appropriate conditions (different from the high flash point terpene) for inclusion as a second strip in the present film.

A further advantage of the present application is the provision of a film comprising distinct strips which provides flexibility with respect to the delivery of the terpenes and/or any other active ingredients. For example, the present film may be provided with strips that exhibit different dissolution rates, or exhibit different adhesion properties, to provide staged release of the active ingredients. Additionally, the provision of strips in the present film provides a means to deliver different terpenes or combinations of terpenes which are known to provide different effects and/or tastes when mixed in various combinations and/or quantities. Thus, a certain terpene combination may be provided in one strip to achieve a desired effect/taste, while another terpene combination may be included in a separate strip to achieve a distince effect/taste without modifying the effect of the first terpene strip.

In another aspect of the invention, a film is provided comprising a coating of a buccal and/or sublingual mucosal absorption enhancer. A buccal and/or sublingual mucosal absorption enhancer is a compound that functions to increase absorption of the film and its content by stimulating buccal and/or sublingual mucosal absorption. Examples of suitable such absorption enhancers include, but are not limited to, dextro-limonene, nerolidol, alpha-bisabolol, eucalyptol, 23-lauryl ether, aprotinin, azone, taurine, dextran sulfate, cetylpyridinium chloride, lauric acid, menthol, xylitol, oleic acid, polyoxyethylene, polysorbate 80, salts of EDTA, deoxycholate, glycocholate, lauryl sulfate, glycodeoxycholate, salicylate, taurocholate and dodecyl sulphate (e.g. sodium salts), palmitoyl carnitine and alkyl glycosides. For application to a film, the absorption enhancer is encapsulated in a charged powder of maltodextrin, inulin, a-tocopherol, acacia gum, or a mixtures thereof in an amount in the range of about 0.001-5% by wt of the film. The powder is charged by exposure to a high voltage low amperage charge (e.g. 60 kV to 100 kV/25 to 30 microamperes (μA)). The buccal/sublingual absorption enhancer is applied in the charged powder to both sides of the film following its manufacture at a thickness in the range of about 25-200 microns and compressed into the film, for example, by passage between two heated rollers (e.g. heated to about 50 to 98° C.).

In a further aspect, a film is provided comprising an anti-microbial coating, e.g. anti-bacterial, anti-fungal, anti-parasitic coating. The coating may be applied to the film in a manner similar to a buccal/sublingual absorption enhancer coating. Examples of anti-microbial agents that may be coated on the film include, but are not limited to, terpenes such as nerolidol, ocimene, para-cymene, limonene, eucalyptol, citrol, gamma terpinene and geraniol; however, other anti-microbial agents may also be used such as acetic acid, lactic acid, propionic acid, sorbic acid and benzoic acid. The amount of anti-microbial in the powdered coating is in the range of about 0.001 5% by wt of the film, which is applied to the film at a thickness in the range of about 25-200 microns. Preferably the film is coated with nerolidol which has the dual function of buccal absorption enhancer and anti-microbial. The provision of a film with an anti-microbial coating prevents undesirable infection by a user. Since the present films are generally contained in a case, in order to consume the film, it must be removed from the case using fingers, which are not necessarily clean at all times, and then placed in the mouth, providing the opportunity for infection to a user. In some instances, the terpene within the film may function as both the active ingredient and protective anti-microbial agent.

Embodiments of the invention are described in the following specific examples which are not to be construed as limiting.

### EXAMPLE 1

### An Edible Film Having Quick and Slow Dissolving Adjoined Strips

A terpene-containing film comprising 2 adjacent adjoined film strips, a quick dissolving film strip and a slow dissolving film strip, was prepared.

The quick dissolving film was prepared by blending the following dry ingredients: sorbitol (7.3 g/16.73 wt %), sodium alginate (12 g/27.5 wt %) and pullulan (19 g/43.55 wt %). Potable water (100 g) was added to the dry ingredients at 68° C. and mixed with a high shear mixer for 30 min. to form a slurry. Glycerin (1.45 g/3.32 wt %) was added to the slurry which was continuously blended at 50 rpm for 25 min. Limonene was also added to the slurry in an amount of 2.5 g/6 wt %. Once blended, the slurry was allowed to rest for 2 hours.

The slow dissolving film was prepared by blending the following dry ingredients: sorbitol (7.3 g/19.16 wt %), hydroxypropyl methylcellulose (12 g/31.49 wt %), carrageen (5 g/13.12 wt %) and maltodextrin (9 g/23.62 wt %). Potable water (100 g) was added to the dry ingredients at 68° C. and mixed with a high shear mixer for 30 min. to form a slurry. Glycerin (1.45 g/3.81 wt %) was added to the slurry which was continuously blended at 50 rpm for 25 min. Humulene was also added to the slurry in an amount of 2.5 g/6 wt %. Once blended, the slurry was allowed to rest for 2 hours.

The resulting slurries were prepared into a film by being drawn through an extruder adapted to extrude the 2 films adjacently and conjoined onto a moving mylar web which was passed through a series of heating (45° C. to 95° C.) and cooling cycles to obtain a film having a 9% homogeneous moisture level, a quick dissolving terpene strip and a slower dissolving terpene strip.

The quick-dissolving strip was determined to have a dissolve rate of 10 seconds and the slow-dissolving strip had a dissolve rate of 65 seconds utilizing EI QC 019.

### EXAMPLE 2

### An Edible Film Having a Terpene and Plant Extract Adjoined Strips

A film comprising 2 adjacent adjoined film strips, an oregano-containing strip and an alpha bisabolol-containing strip, was prepared.

The film was prepared as above by combining dry ingredients: sorbitol (7.3 g/15.60% by wt), starch (12 g/25.64% by wt), sodium alginate (10 g/21.37% by wt), pullulan (9 g/19.23% by wt), sorbitan monostearate (1.45 g/3.10% by wt) and polysorbate 60 (0.85 g/1.82% by wt). The dry blend was mixed with water (100 g) as previously described to form a slurry which was divided.

Oregano (1.7 g/3.6% by wt) and alpha bisabolol (0.28 g/0.6% by wt) were added to separate slurries pre-extrusion, blended and then rested for 2 hours.

The resulting slurries were prepared into a film by being drawn through an extruder adapted to extrude the 2 films adjacently and conjoined onto a mylar web which was passed through a series of heating (45° C. to 95° C.) and cooling cycles to obtain a film having a 9% homogeneous moisture level.

### EXAMPLE 3

### An edible Film Coated with an Anti-Microbial Agent

A quick dissolving film strip was prepared as above by combining the following dry ingredients: sorbitol (7.3 g/15.60% by wt), starch (12 g/25.64% by wt), sodium alginate (10 g/21.37% by wt), pullulan (9 g/19.23% by wt), sorbitan monostearate (1.45 g/3.10% by wt) and polysorbate 60 (0.85 g/1.82% by wt). The dry blend was mixed with water (100 g water) as previously described to form a slurry which was divided. Ocimene (0.400 mg) and limonene (0.450 g) were added to separate slurries pre-extrusion, each were blended and then rested for 2 hours.

The resulting slurries were prepared into a film by being drawn through an extruder adapted to extrude the film onto a mylar web which was passed through a series of heating (45 to 90° C.) and cooling cycles to obtain a film having a 9% homogeneous moisture level.

The film was then coated with a charged antibacterial/antifungal agent (Geraniol) that was infused into a starch powder in an amount of 4.8% by wt, and charged by exposure to a high voltage low amperage charge (e.g. 60 kV to 100 kV/25 to 30 microamperes (μA) for 1.5 s. The anti-microbial charged powder was applied to both sides of the film at a thickness in the range of about 25-200 microns and compressed into the film by passage between two heated rollers (e.g. heated to about 50 to 98 degrees ° C.).

### EXAMPLE 4

### An Edible Strip Having Linalool and Myrcene Adjoined Strips

A film comprising 2 adjacent adjoined film strips, a linalool-containing strip and a myrcene-containing strip, was prepared as above by combining the dry ingredients: sorbitol (7.3 g/15.60% by wt), starch (12 g/125.64% by wt), sodium alginate (10 g/21.37% by wt), pullulan (9 g/19.23% by wt), sorbitan monostearate (1.45 g/3.10% by wt) and polysorbate 60 (0.85 g/1.82% by wt). The dry blend was mixed with water (100 g water) as previously described to form a slurry.

Linalool (1.6 mg/0.36% by wt) and myrcene (0.8 mg/0.18% by wt) were added to separate slurries pre-extrusion, blended and then rested for 2 hours.

The resulting slurries are prepared into a film by being drawn through an extruder adapted to extrude the 2 films adjacently and conjoined onto a mylar web which was passed through a series of heating (45 to 90° C.) and cooling cycles to obtain a film having a 9% homogeneous moisture level.

### EXAMPLE 5

### An Edible Film Coated with a Buccal/Sublingual Mucosal Absorption Enhancer

A quick dissolving film strip was prepared as above by combining the following dry ingredients: sorbitol (7.3 g/15.60% by wt), starch (12 g/25.64% by wt), sodium alginate (10 g/21.37% by wt), pullulan (9 g/19.23% by wt), sorbitan monostearate (1.45 g/3.10% by wt) and polysorbate 60 (0.85 g/1.82% by wt). The dry blend was mixed with potable water (100 g water) as previously described to form a slurry. Ocimene (0.400 mg) and eucalyptol (0.450 g) were separately added to slurry pre-extrusion, each were blended and then rested for 2 hours

The resulting slurries were prepared into a film by being drawn through an extruder adapted to extrude the film onto a moving belt, drum or mylar web, which was then passed through a series of heating (45 to 90° C.) and cooling cycles to obtain a film having a 9% homogeneous moisture level.

The film was then coated with a charged buccal/sublingual absorption enhancer (limonene) that was infused into a starch powder in an amount of 4.8% by wt, and charged by exposure to a high voltage low amperage charge (e.g. 60 kV to 100 kV/25 to 30 microamperes (μA) for 1.5 s. The buccal absorption enhancer charged powder was applied to both sides of the film at a thickness in the range of about 25-200 microns and compressed into the film by passage between two heated rollers (e.g. heated to about 50 to 98 degrees ° C.).

The film was then coated with a charged buccal absorption enhancer (limonene) that was infused into a starch powder in an amount of 4.8% by wt, and charged by exposure to a high voltage low amperage charge (e.g. 60 kV to 100 kV/25 to 30 microamperes (μA) for 1.5 s). The buccal absorption enhancer charged powder was applied to both sides of the film at a thickness in the range of about 25-200 microns and compressed into the film by passage between two heated rollers (e.g. heated to about 50 to 98 degrees ° C.).

1. An edible film comprising at least one terpene or terpenoid, at least one film-forming agent, at least one surfactant or emulsifier, and a plasticizer, wherein the film comprises at least two adjacent conjoined strips, each strip having a distinct functionality.

2. The edible film of claim 1, wherein the terpene is selected from the group consisting of a hemiterpenes, monoterpenes, sesquiterpenes, diterpenes, sesterterpenes, triterpenes, sesquarterpenes, tetraterpenes, hydroxylated terpenes, oxygenated terpenes, and mixtures thereof.

3. The edible film of claim 1, wherein the terpene is selected from the group consisting of: myrcene, beta caryophyllene, pinene, limonene, terpinolene, humulene, nerolidol, linalool, ocimene, guaiol, bisabolol, alpha phellandrene, cadinene, camphene, camphor, citral, citronellol, delta 3-carene, eucalyptol, eugenol, gamma terpinene, geraniol, humulene, nerol, nerolidol, ocimene, paracymene, phytol, pulegone, terpineol, valencene, prenol, isovaleric acid, geraniol, an iridoid, farnesene, farnesol, B-bisabolene, B-curcimene, lanceol, juvabione, humbertiol, cafestol, kahweol, cembrene, taxadiene, geranylfarnesol, squalene, ferrugicadiol, tetraprenylcurcumene, acyclic lycopene, monocyclic gamma-carotene, bicyclic alpha- or beta-carotene, oxides thereof, hydroxylated analogues thereof or mixtures thereof.

4. The edible film of claim 1, comprising a terpene or terpenoid in an amount in the range of 0.01% to 30% by wt.

5. The edible film of claim 1, comprising a film forming agent in an amount ranging from 30 to about 80 wt %, a surfactant or an emulsifier in an amount ranging from 0.5 to 15% by wt, and a plasticizer in an amount ranging from 2 to 18% by wt.

6. The edible film of claim 1, wherein the film forming agent is selected from the group consisting of pullulan, sodium alginate, carrageenan, methyl cellulose, ethyl cellulose, hydroxypropylmethyl cellulose, hydroxypropyl cellulose, corn starch, pectin, chitin, chitosan, collagen, gelatin, zein, gluten, cassava starch, glucan and mixtures thereof.

7. The edible film of claim 6, additionally comprising one or more agents that facilitate film forming selected from the group consisting of hydroxyethyl cellulose, polyvinyl pyrrolidone, carboxymethyl cellulose, polyvinyl alcohol, polyethylene glycol, polyacrylamides, polyvinyl alcohol, polyethylene glycol, glycolide, polylactic acid, polylactide, cellulose gum, xanthan gum, tragacanth gum, guar gum, acacia gum, arabic gum, polyacrylic acid, sodium methacrylic acid polymers, methacrylic acid copolymers, acrylic acid polymers, acrylic acid copolymers, methylmethacrylate copolymer, carboxyvinyl polymer, amylose, high amylose starch, casein, algin, maize, chondrus crispus, crosspovidone (CPV), polyvinylpyrrolidone (PVP), hydroxymethylcellulose, gum tragacanth, gum karaya, glycogen, phytoglycogen, welan gum, polysaccharides, hydroxypropylate, dextrin, maltodextrin, soy protein isolate, whey protein isolate, beeswax, carnauba wax, fungal exopolysaccharides and rapeseed.

8. The edible film of claim 1, comprising a surfactant selected from the group consisting of glycerol, glycerin, propylene glycol, sucrose fatty acid ester, sorbitan monostearate, sorbitol-based emulsifier, lecithin, modified cellulose gum (CMC), ceratonia gum, locust bean gum, sodium lauryl sulfate (SLS) and mixtures thereof.

9. The edible film of claim 1, comprising an emulsifier selected from the group consisting of poly-oxyethylene, sorbitan monostearate, lecithin, stearate salt, calcium stearoyl lactylate (CSL), glyceryl monostearate, mono propyleneglycol, sorbitan monooleate, sorbitol-based emulsifier, monoglyceride, glycerol triacetate and mixtures thereof.

10. The edible film of claim 1, comprising a plasticizer selected from the group consisting of monostearate palm oil, lethicin, sorbitan monostearate, glycerin, glycerin fatty acid ester, triacetin, pre-gelatinized starch, sorbitol, a medium chain mono- or diglyceride, castor oil, malic acid, polyethylene glycol (PEG), glycerol, palm oil and mixtures thereof.

11. The edible film of claim 1, additionally comprising a sweetener and/or a flavouring agent.

12. The edible film of claim 1, comprising an additional active ingredient is a nutraceutical or a pharmaceutical agent in an amount in the range of 0.01% to 15% by wt.

13. The edible film of claim 1, wherein each strip exhibits a different rate of dissolution.

14. The edible film of claim 1, comprising a first strip made of a film forming agent selected from pullulan, sodium alginate, and mixtures thereof, and a second strip made of a film forming agent selected from hydroxypropyl methylcellulose, carrageen, and mixtures thereof.

15. The edible film of claim 1, additionally comprising a buccal and/or sublingual mucosal absorption enhancer.

16. The edible film of claim 15, wherein the buccal and/or sublingual mucosal absorption enhancer is selected from 23-lauryl ether, aprotinin, azone, taurine, dextran sulfate, cetylpyridinium chloride, lauric acid, menthol, xylitol, oleic acid, polyoxyethylene, polysorbate 60 or 80, salts of EDTA, deoxycholate, glycocholate, lauryl sulfate, glycodeoxycholate, salicylate, taurocholate or dodecyl sulphate, palmitoyl carnitine, an alkyl glycoside, alpha-limonene, nerolidol, limolene and mixtures thereof

17. The edible film of claim 1, comprising an anti-oxidant.

18. The edible film of claim 17, wherein the antioxidant is selected from sodium thiosulfate, ascorbic acid, fatty acid esters of ascorbic acid, alpha-tocopherol, propyl gallate, erythorbic acid, sodium erythorbate, citric acid, tocopherols, alkyl-substituted paraben or salts thereof, and mixtures thereof.

19. The edible film of claim 1, coated with a buccal and/or sublingual mucosal absorption enhancer.

20. The edible film of claim 1, coated with an anti-microbial agent.

21. An edible film comprising at least one terpene or terpenoid, at least one film-forming agent, at least one surfactant or emulsifier, and a plasticizer, wherein the film is coated with a buccal and/or sublingual mucosal absorption enhancer or an anti-microbial agent or both.

22. The film of claim 21, wherein the buccal and/or sublingual mucosal absorption enhancer is selected from 23-lauryl ether, aprotinin, azone, taurine, dextran sulfate, cetylpyridinium chloride, lauric acid, menthol, xylitol, oleic acid, polyoxyethylene, polysorbate 60 or 80, salts of EDTA, deoxycholate, glycocholate, lauryl sulfate, glycodeoxycholate, salicylate, taurocholate or dodecyl sulphate, palmitoyl carnitine, an alkyl glycoside, alpha-limonene, nerolidol, limolene and mixtures thereof

23. The film of claim 21, wherein the buccal and/or sublingual absorption enhancer is encapsulated in a charged powder of maltodextrin, inulin, α-tocopherol, acacia gum, or a mixtures thereof.

24. The film of claim 21, wherein the anti-microbial agent is an anti-bacterial agent, an anti-fungal agent, an anti-viral agent, an anti-parasitic agent, or a mixture thereof.

25. The film of claim 21, wherein the anti-microbial agent is selected from nerolidol, ocimene, para-cymene, limonene, eucalyptol, citrol, gamma terpinene, geraniol and mixtures thereof.

26. The film of claim 21, wherein the anti-microbial agent is applied as coating which is 25-200 microns thick and comprising 0.001-5% by wt of the anti-microbial agent.

27. The edible film of claim 1, comprising 2 or more terpenes, each terpene in a separate strip.

28. The edible film of claim 1, comprising 2 or more terpenes that mimic the terpene content of a Cannabis plant.

29. The edible film of claim 1, comprising a cannabinoid.

30. The edible film of any one of claims 1-29, wherein the terpene or terpenes are obtained from non-Cannabis plant sources.

31. The edible film of claim 1, wherein the adjacent strips are in a side-by-side arrangement.
